(19) United States
(12) Patent Application Publication
(10) Pub. No.: US 2024/0269437 A1
(43) Pub. Date: Aug. 15, 2024

(54) CATHETER ASSEMBLY WITH NEEDLE TIP PROTECTION AND PASSIVE BLOOD CONTROL

(71) Applicant: SMITHS MEDICAL ASD, INC., PLYMOUTH, MN (US)

(72) Inventors: Christopher D Roehl, New Hartford, CT (US); Thomas T Koehler, Pelzer, SC (US)

(73) Assignee: SMITHS MEDICAL ASD, INC., PLYMOUTH, MN (US)

(21) Appl. No.: 18/571,319

(22) PCT Filed: Jul. 25, 2022
(86) PCT No.: PCT/US2022/038120

§ 371 Date: Dec. 18, 2023

Related U.S. Application Data
(60) Provisional application No. 63/227,520, filed on Jul. 30, 2021.

(30) Foreign Application Priority Data
Jul. 30, 2021 (US) 63227520

(51) Int. Cl. A61M 25/06; A61M 25/00

(52) U.S. Cl. CPC A61M 25/0618; A61M 25/0097; A61M 25/0606; A61M 25/0637

(57) ABSTRACT

A safety catheter assembly that may be a closed system catheter (CSC) has a seal member proximal a septum in the catheter hub. In a first embodiment, the seal member may be a cylindrical sleeve whereto a locking portion of a needle clip is positioned to fixedly hold the needle clip inside the catheter hub in a ready state. In a second embodiment, the needle clip is housed in a substantially cylindrical seal member. The seal member with the needle clip therein and the catheter hub are fixedly coupled together in the ready state. In a third embodiment, a retainer member at the catheter hub couples the needle clip to the catheter hub in the ready state and the seal member is distal the retainer member. In all the embodiments, the seal member may act as a reservoir for fluid in the interior cavity of the catheter hub that may back flow past the septum.

### FIELD OF THE INVENTION

The present invention relates to safety catheter assembly devices that have passive needle tip protection mechanisms that couple the needle housing to the catheter hub of the devices and shield the sharp distal tip of the needle of the devices from when the devices are in the ready state to the disposal of the devices after use. Passive blood control mechanism is provided in the devices.

### BACKGROUND OF THE INVENTION

A conventional catheter assembly typically has a catheter hub and a catheter tube extending distally thereof, and a needle assembly mounted together in an over-the-needle fashion. The needle assembly typically includes a needle hub or support and a needle cannula extending distally from the needle hub. A closed system catheter (CSC) is a catheter assembly that may include a sideport at the catheter hub where a tubing from the sideport may be coupled to a fluid store device such as a syringe or a fluid pump. In a ready to use position or a ready state, a needle cannula, or simply needle, attached to a needle hub of the needle assembly extends through the catheter tube to expose its sharp distal tip distally beyond the distal end of the catheter tube. The sharp distal tip of the needle is used to penetrate the tissue of the patient for insertion of the catheter tube within the vasculature system, for example the vein or artery, of a patient. Once the catheter tube is disposed correctly within the vasculature, the needle is withdrawn from the catheter assembly so that a fluid path is established between the fluid store and the vein or artery of the patient, via the sideport and the interior cavity of the catheter hub if the catheter assembly is a CSC assembly. For a catheter assembly where there is no sideport, a fluid path is established between the vein or artery of the patient and a fluid store or tubing that has a connector, most likely a luer, that couples to the opened proximal end of the catheter hub. A needle tip protector may be provided in the catheter assembly to capture at least the sharp distal tip of the needle, if not the entire needle, after use.

The opened proximal end of the catheter hub typically is adapted to receive a male luer taper into the interior cavity of the catheter hub to establish a fluid connection between the patient's vasculature and the luer taper. The proximal end may also be provided with external ears, flange or the like to secure the luer taper in the catheter hub, for example when the luer taper is coupled with a male luer lock collar or nut to form part of a male luer lock connector of an administration set or the end of a syringe, or the like. Under normal conditions, after withdrawal of the needle and before a luer taper is inserted into the catheter hub, blood immediately starts flowing through the catheter tube into the interior cavity of the catheter hub. For a CSC assembly, a septum may be provided in the catheter hub to prevent the blood from back flowing to the tubing extending from the port extending from the side of the catheter hub.

In use, the distal tip of the needle cannula, which extends beyond the distal end of the catheter, is inserted into the vein or artery of the patient. Once the catheter is correctly placed, the needle cannula is removed. The distal tip of the needle, insofar as it was inserted into for example the vein of the patient, is exposed to the blood of the patient. Blood therefore will most likely adhere to the distal portion of the needle. There is always the chance that the contaminated blood adhered to the needle would be exposed to the environment. It is therefore desirable that the excess blood be maintained within the catheter hub as the needle is withdrawn relative to the catheter hub, and that the contaminated distal tip of the needle be prevented from being exposed to the environment when the needle is removed from the catheter hub for disposal.

### SUMMARY OF THE PRESENT INVENTION

The present invention safety catheter assembly encompasses a number of embodiments. A first embodiment has a catheter hub configured to have a length sufficient to house a seal member, and also a septum in the case the catheter assembly is a CSC assembly, as well as a needle tip protector. The seal member may be an elastomeric sleeve that has a substantially cylindrical like configuration including a circumferential wall defining a through bore. There may be a membrane integral in the sleeve to separate the seal member into a proximal section and distal section. The proximal section may have a length that is shorter than the distal section, so long as the length is sufficient to coact with a locking mechanism of the needle tip protector. The locking mechanism may be opposing catches or bumps at respective arms of the needle tip protector that press against the wall of the seal member when the barriers of the needle clip are biased by a needle passing through the needle clip in the catheter hub in a ready to use position or a ready state. The seal member is positioned in place in the catheter hub by means of respective catch mechanisms at the outer surface of the wall of the seal member and the inner surface of the wall of the catheter hub.

For a CSC assembly that has a sideport hub integrated to the catheter hub, a septum is positioned inside the catheter hub proximal the opening that establishes a fluid communication path between the interior cavity of the catheter hub distal the septum and the bore of the sideport. With the needle passing through the catheter hub and biasing the barriers of the needle clip in a transverse direction away from the longitudinal axis of the catheter hub, the opposing bumps associated with the respective arms move in unison with the barriers to press against the wall of the seal member to frictionally hold the needle clip inside the catheter hub. After the catheter that extends distally from the catheter hub is correctly placed into the vasculature for example a vein of the patient, the needle is withdrawn from the catheter and then from the catheter hub. As the distal tip of the needle is withdrawn past the septum, fluid in the interior cavity of the catheter hub distal the septum might back flow into the chamber defined by the distal section of the seal member. When the distal tip of the needle is withdrawn past the membrane of the seal member, the elastomeric membrane reseals to close the chamber so that the chamber acts as a reservoir to store the back flowed fluid. When the distal tip of the needle is drawn into the needle clip and is proximal the barriers of the needle clip, the barriers are no longer being biased by the needle. Accordingly, the respective arms of the barriers would return to their natural position such that the opposing bumps of the arms are released from the wall at the proximal section of the seal member. As a result, the needle clip, with the distal portion of the needle captured therein, may be removed from the catheter hub. The needle housing that has the needle hub to which the proximal end of the needle is attached may have a distal housing or collar that houses the needle clip when it separates from the catheter hub, if a portion of the needle clip extends out of the catheter hub in the ready state.

Another embodiment of the instant invention has the needle clip completely housed inside a seal member. In this embodiment, the seal member is made of elastomeric material and has a substantially hollow cylindrical body including a base and a front that may have respective apertures. In the ready state, the seal member is housed in the interior cavity of the catheter hub proximal to a septum. In a CSC assembly where the catheter hub has an integral sideport, the septum is positioned in the catheter hub proximal an opening that connects the bore for the sideport with the interior cavity of the catheter hub. When a needle extends through the catheter hub to the catheter that extends from the distal end of the catheter hub, the barriers at the arms of the needle clip are biased transversely away from the longitudinal axis of the catheter hub such that opposing bumps at the arms press against corresponding portion of the seal member. The being pressed against portions of the seal member in turn press against a catch mechanism for example an internal groove at the inner wall of the catheter hub such that the needle clip and the seal member are fixedly attached to the catheter hub when the catheter assembly is in the ready state.

After use, the needle is withdrawn proximally relative to the catheter hub such that the distal tip of the needle is removed from the catheter, the septum and the seal member, and eventually is captured in the needle clip so that the barriers are no longer biased transversely away from the longitudinal axis of the catheter hub by the needle. As a result, the opposing bumps that follow the movement of the barriers would return to their natural position by moving toward the center of the needle clip and be released from the internal groove at the inner wall of the catheter hub. Thereafter, the seal member with the needle clip housed therein may be removed from the catheter hub. As the front end of the seal member is in abutment with the proximal surface of the septum, the fluid that may back flow from the interior cavity of the catheter hub distal the septum would be collected in the seal member, which acts as a reservoir for the back flow fluid, before the seal member and the needle clip are removed from the catheter hub. The elastomeric septum would have resealed by the time the distal tip of the needle is captured in the needle clip to prevent further back flow of fluid from the interior cavity of the catheter hub distal the septum.

A further embodiment has a catheter hub that has a retainer member at the opened proximal end of the catheter hub. The retainer member has an opening dimensioned to prevent a needle clip that has its barriers being biased by a needle from passing through. Positioned in the interior cavity of the catheter hub are a distal septum and a proximal septum that may be a seal member. The distal septum is made from an elastomeric material and may have an aperture for a needle to pass through. In the case of a CSC assembly, the distal septum is positioned in the interior cavity of the catheter hub proximal the opening that establishes the fluid through path between the sideport and the interior cavity distal the septum. The proximal septum may also be made from an elastomeric material, and is a substantially cylindrical hollow member that has an opened distal end in abutment with the proximal surface of the distal septum. An opening may be provide at the proximal base of the proximal septum. Instead of an opening, one or more resealable slits may be provided at the proximal base of the proximal septum to assist the needle to pierce through the proximal base.

When the needle is withdrawn, with the distal tip of the needle proximal the proximal septum, the slits at the proximal base reseal so the chamber defined by the proximal septum acts as a reservoir for fluid that may have back flowed through the distal septum from the interior cavity of the catheter hub distal the distal septum. For this embedment, the needle clip is the same as the earlier embodiments, that is, the opposing bumps are connected to and move in association the movement of the barriers of the needle clip. In the ready state where the needle passes between the barriers, the barriers are biased away from the center of the needle clip such that the opposing bumps expand outwardly to effect a cross dimension that is greater than the dimension of the opening of the retainer member. As a result, the needle clip is not removable from catheter hub. After use, the needle is withdrawn proximally relative to the catheter hub. When the distal tip of the needle is trapped in the needle clip, the opposing bumps would move toward the center of the needle clip following the movement of the barriers to their natural position. As a result, the cross dimension of the opposing bumps is smaller than the dimension of the opening of the retainer member so that the needle clip is removable from the catheter hub. To prevent exposure of the needle clip during use, a collar is provided at the distal end of the needle housing to encircle the needle clip. The needle clip is non-removably housed in the collar.

The present invention is thus directed to a catheter assembly comprising:
a catheter hub having a body defined by a wall having a distal end and an opened proximal end, and an interior cavity defined by an inner surface of the wall between the distal end and the opened proximal end;
a catheter having a distal end extending from the distal end of the catheter hub along a longitudinal axis to establish a passage to the interior cavity;
a septum positioned in the interior cavity of the catheter hub;
a needle housing having a needle hub and a distal portion having an opened distal end;
a needle having a proximal end attached to the needle hub and a sharp distal tip extending away from the distal end of the needle housing;
wherein the needle extends into the interior cavity of the catheter hub and slidably extends along the catheter with its distal tip extending beyond the distal end of the catheter and the distal portion of the needle housing adjacent to the proximal end of the catheter hub when the catheter assembly is in a ready state;
a needle tip protector slidably positioned along the needle, the needle tip protector having a base and opposing arms extending distally from the base to respective opposing apexes having corresponding opposing bumps and corresponding barriers downward of the apexes, the opposing bumps movable bi-directionally transversely to the longitudinal axis;
a sleeve member fixedly engaged to and in circumferential contact with the inner surface of the wall of the catheter hub;
wherein when the catheter assembly is in the ready state, the distal bumps of the opposing arms of the needle tip protector are positioned within the interior cavity of the catheter hub proximal to the septum, the needle passing between the barriers and biasing the barriers in a direction away from the longitudinal axis to cause the opposing bumps to be in frictional contact with the sleeve member; and
wherein when the needle is withdrawn from the catheter and the distal tip of the needle moves proximally past the barriers so that the barriers are no longer biased by the needle, the opposing bumps move away from the sleeve member toward the longitudinal axis to disengage the needle tip protector from the sleeve member such that the needle tip protector is separable from the catheter hub.

The present invention is further directed to a catheter assembly comprising:
a catheter hub having a wall defining a body having an interior cavity defined by an inner surface of the wall between a distal end and an opened proximal end, a side port hub extending from the body, an opening at the wall connecting a passage in the side port hub with the interior cavity, a catheter having a distal end extending from the distal end of the catheter hub along a longitudinal axis to establish a fluid passage to the interior cavity;
a septum positioned in the interior cavity of the catheter hub proximal the opening at the wall;
a needle housing having a needle hub and a distal portion having an opened distal end;
a needle having a proximal end attached to the needle hub and a sharp distal tip extending away from the distal end of the needle housing, the needle extending into the interior cavity of the catheter hub, piercing past the septum and slidably extends along the catheter with its distal tip extending beyond the distal end of the catheter in a ready state;
a sleeve member fixedly engaged to and in circumferential contact with the inner surface of the wall of the catheter hub, a distal end of the sleeve member in contact with a proximal surface of the septum;
a needle tip protector slidably positioned along the needle, the needle tip protector having a base and opposing arms extending distally from the base to respective opposing apexes having corresponding opposing bumps and corresponding barriers transverse of the apexes toward the longitudinal axis, the opposing bumps movable bi-directionally transversely to the longitudinal axis;
wherein, when the catheter assembly is in the ready state, the distal bumps of the opposing arms of the needle tip protector are positioned within the sleeve member, the needle passing between the barriers and biasing the barriers in a direction away from the longitudinal axis such that the opposing bumps are biased into frictional contact with the sleeve member.

Another embodiment of the present invention is directed to a catheter assembly, comprising:
a catheter hub having a body defined by a wall having a distal end and an opened proximal end, and an interior cavity defined by an inner surface of the wall between the distal end and the opened proximal end;
a catheter having a distal end extending from the distal end of the catheter hub along a longitudinal axis to establish a passage to the interior cavity;
a needle having a proximal end attached to a needle hub and a sharp distal tip, the needle extends into the interior cavity of the catheter hub and slidably extends along the catheter with its distal tip extending beyond the distal end of the catheter when the catheter assembly is in a ready state;
a needle tip protector slidably positioned along the needle, the needle tip protector having a base and opposing arms extending distally from the base to respective opposing apexes having corresponding opposing bumps and corresponding barriers downward of the apexes, the opposing bumps movable bi-directionally transversely to the longitudinal axis;
a seal member having a substantially hollow cylindrical body extending from a proximal base having a base aperture to a distal front having a front aperture, the distal and front apertures in alignment with each other;
wherein the needle tip protector is housed in the seal member such that the needle passes through the base aperture, between the barriers of the needle tip protector so as to bias the barriers in a direction away from the longitudinal axis such that the opposing bumps are moved to extend given opposing portions of the cylindrical body, and through the front aperture when the catheter assembly is in the ready state.

The embodiment of the present invention is further directed to a catheter assembly, comprising:
a catheter hub having a body defined by a wall having a distal end and an opened proximal end, and an interior cavity defined by an inner surface of the wall between the distal end and the opened proximal end, a catheter having a distal end extending from the distal end of the catheter hub along a longitudinal axis to establish a passage to the interior cavity;
a seal member having a hollow body housing a needle tip protector positioned in the interior cavity, the body connecting a proximal base having a base aperture and a distal front having a front aperture, the needle tip protector including a base having an opening and opposing arms extending distally from the base to respective opposing apexes having corresponding opposing bumps and corresponding barriers downward of the apexes, the opposing bumps movable bi-directionally transversely to the longitudinal axis;
wherein the seal member is fixedly held in the interior cavity of the catheter hub when a needle having a sharp distal tip passes through the base aperture of the seal member and the opening at the base of the needle tip protector, passes between the barriers of the needle tip protector such that the barriers are biased in a direction away from the longitudinal axis to move the opposing bumps to extend given opposing portions of the body into locking engagement with a locking mechanism at the inner surface of the wall of the catheter hub.

Yet another embodiment of the present invention is directed to a catheter hub for a catheter assembly, comprising:
a catheter hub having a body along a longitudinal axis defined by a wall having a distal end and an opened proximal end, and an interior cavity defined by an inner surface of the wall between the distal end and the opened proximal end, a catheter having a distal end extending from the distal end of the catheter hub along a longitudinal axis to establish a passage to the interior cavity;
a septum positioned in the interior cavity of the catheter hub; and
a seal member having a body fixedly attached to the interior cavity of the catheter hub, the seal member having a proximal base that includes an aperture dimensioned to enable a needle to be in circumferential abutment with the aperture when passing therethrough and an opened distal end in abutment with a proximal surface of the septum.

The another embodiment of the present invention is yet further directed to a catheter assembly, comprising:
a catheter hub having a body defined by a wall having a distal end and an opened proximal end, and an interior cavity defined by an inner surface of the wall between the distal end and the opened proximal end, a catheter having a distal end extending from the distal end of the catheter hub along a longitudinal axis to establish a passage to the interior cavity;
a needle having a proximal end attached to a needle hub and a sharp distal tip, the needle extends into the interior cavity of the catheter hub and slidably extends along the catheter with its distal tip extending beyond the distal end of the catheter when the catheter assembly is in a ready state;
a septum positioned in the interior cavity of the catheter hub;
a seal member having a body fixedly attached to the interior cavity of the catheter hub, the seal member having a proximal base that includes an aperture dimensioned to enable a needle to be in circumferential abutment with the aperture when passing therethrough and an opened distal end in abutment with a proximal surface of the septum; and
a needle tip protector slidably positioned along the needle in the interior cavity proximal to the seal member in the ready state, the needle tip protector including a base having an opening through which the needle passes and opposing arms extending distally from the base to respective opposing apexes having corresponding opposing bumps, the arms including respective barriers each angled away from its corresponding apex toward the longitudinal axis, the opposing bumps movable bi-directionally transversely to the longitudinal axis to cause the respective barriers to move relative to the longitudinal axis, the barriers biased by the needle in the ready state.

Another embodiment of the present invention is directed to a catheter assembly, comprising:
a catheter hub having a body defined by a wall having a distal end and an opened proximal end, and an interior cavity defined by an inner surface of the wall between the distal end and the opened proximal end, a catheter having a distal end extending from the distal end of the catheter hub along a longitudinal axis to establish a passage to the interior cavity;
a needle having a proximal end attached to a needle hub and a sharp distal tip, the needle extends into the interior cavity of the catheter hub and slidably extends along the catheter with its distal tip extending beyond the distal end of the catheter when the catheter assembly is in a ready state;
a septum positioned in the interior cavity of the catheter hub;
a needle tip protector slidably positioned along the needle in the interior cavity proximal to the seal member in the ready state, the needle tip protector including a base having an opening through which the needle passes and opposing arms extending distally from the base to respective opposing apexes having corresponding opposing bumps, the arms including respective barriers each angled away from its corresponding apex toward the longitudinal axis, the opposing bumps movable bi-directionally transversely to the longitudinal axis to cause the respective barriers to move relative to the longitudinal axis, the barriers biased by the needle in the ready state; and
a retainer member fixedly attached to the opened proximal end of the catheter hub, the retainer member having an opening dimensioned to prevent the needle tip protector from separating from the catheter hub when the barriers of the needle tip protector are being biased by the needle.

### BRIEF DESCRIPTION OF THE FIGURES

The present invention will become apparent and the invention itself will be best understood with reference to the following description of the invention taken in conjunction with the accompanying drawings, wherein:

FIG. 1 is an overall view of a first embodiment of the catheter assembly of the present invention in a ready state;

FIG. 2 is a cross-sectional view of the catheter hub and its relationship to the seal member and needle clip;

FIG. 3 is a cross-sectional perspective view of FIG. 2;

FIG. 4 is a cross-sectional perspective view of FIG. 3 but with the needle tip portion of the needle captured in the needle clip;

FIG. 5 is an overall view showing the needle clip having been separated from the catheter hub;

FIG. 6 is an enlarged cross-sectional view showing the needle clip separated from the catheter hub;

FIG. 7 is another embodiment of the catheter assembly of the present invention in the ready state;

FIG. 8 is a cross-sectional view of the needle clip housed in a seal member positioned in the catheter hub of the catheter assembly;

FIG. 9 is a cross-sectional perspective view of FIG. 8;

FIG. 10 is a cross-sectional view of FIG. 9 but with the distal tip portion of the needle shown captured in the needle clip;

FIG. 11 is a cross-sectional perspective view showing the seal member having the needle clip housed therein separated from the catheter hub;

FIG. 12 show an overall view of the needle clip housed in the seal member separated from the catheter hub assembly in the safe state;

FIG. 13 is an overall view of yet another embodiment of the present invention in its ready state;

FIG. 14 is a cross-sectional view showing the interrelationship among the needle clip, a septum, a seal member, a retainer member at the opened proximal end of the catheter hub and a collar of the needle housing of the FIG. 13 embodiment;

FIG. 15 is an enlarged cross-sectional perspective view of the collar housing the needle clip and the retainer member at the proximal end of the catheter hub;

FIG. 16 is a cross-sectional perspective view showing the collar and the needle clip separated from the catheter hub fitted with the retainer member; and

FIG. 17 is an overall view showing the needle housing and its distal collar separated from the catheter hub of the catheter assembly.

### DETAILED DESCRIPTION OF THE INVENTION

For the discussion below, it should be appreciated that the term “distal”, as used herein, refers to the direction along an axis that lies parallel to a needle cannula of safety catheter assembly, or a closed system catheter assembly (CSC), that is closest to the subject during catheter insertion. Conversely, the term “proximal,” as used herein, refers to the direction lying along the axis parallel to the needle cannula that is further away from the subject when the catheter is inserted into the vein of the subject, opposite to the distal direction. Other terms that may also be used to refer to the distal end and proximal end of the safety catheter device may include “patient end” and “user end”, respectively. Also, for the sake of simplicity, the catheter assembly device, or peripheral intravascular catheter (PIVC) assembly, and the closed system catheter (CSC) assembly may be referred to as “catheter assembly” or “safety catheter”, and the needle tip protector as “safety needle clip” or simply “safety clip” or “needle clip”.

The following US applications and patents, assigned to the same assignee as the present application, relate to safety catheters/catheter assemblies: application Ser. Nos. 15/733,439, 15/435,700, U.S. Pat. Nos. 8,652,104, 10,080,867, 10,548,522 and 10,675,440. The respective disclosures of the '439 and '700 applications and the '104, '867, '522 and '440 patents are incorporated by reference to the disclosure of the present application.

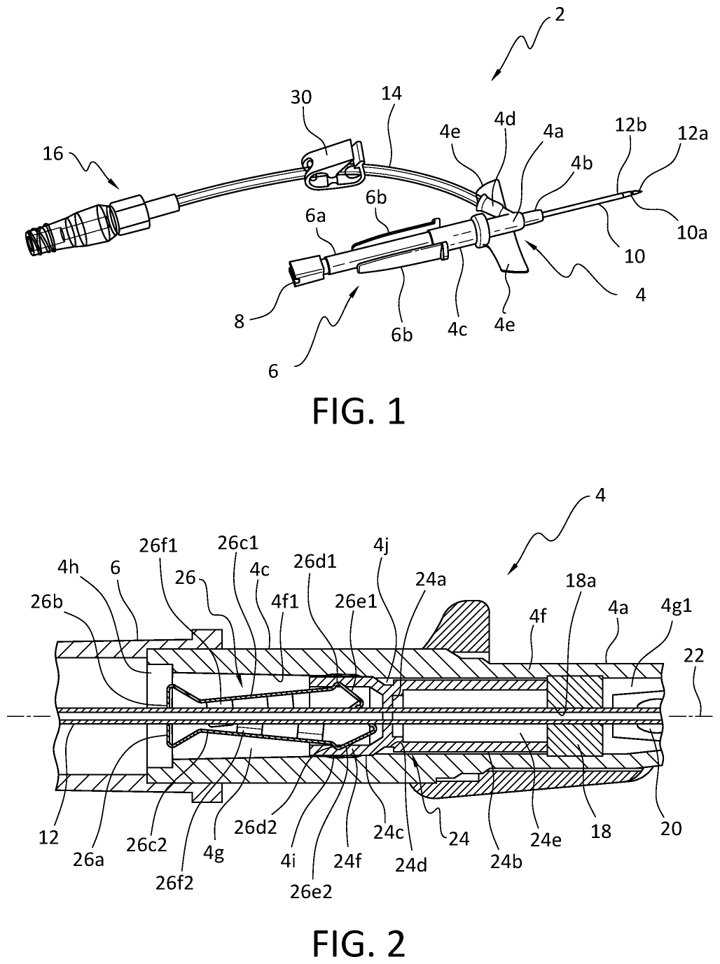

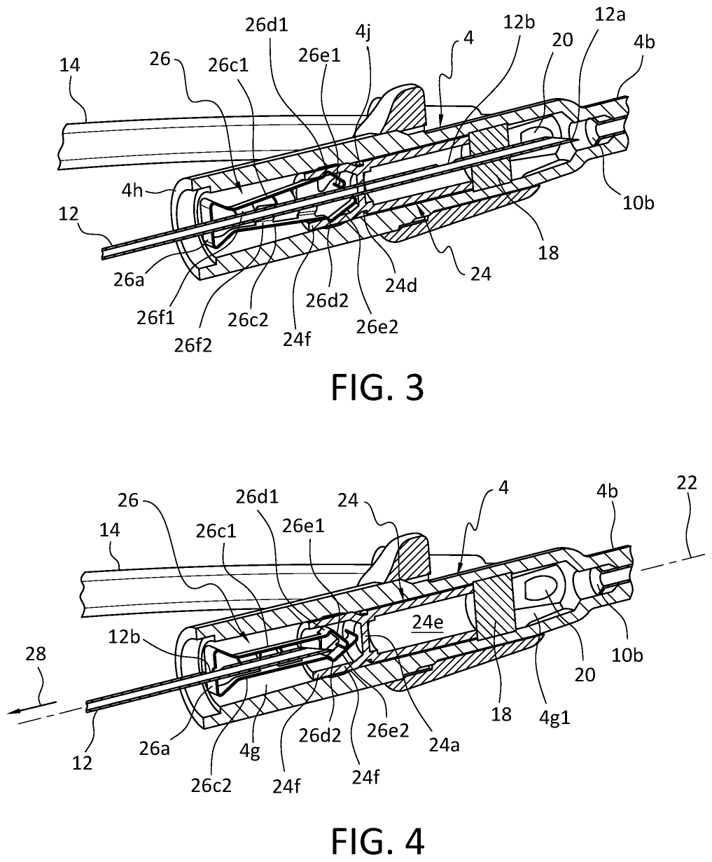

An overall view of the first embodiment of the safety catheter assembly 2 is shown in FIG. 1. As shown, catheter assembly 2 has a catheter hub 4 that has a main body portion 4a, a distal portion 4b, and a proximal portion 4c that has an opened proximal end 4h (FIG. 2). For the closed system catheter (CSC) assembly shown, integrally extending from main body portion 4a is a port that angles toward proximal end. For discussion purpose, the angled port may be referred to as sideport 4d. A tubing 14 extends from the opened end of sideport 4d. The other end of tubing 14 is fitted with a connector 16, which is adapted to couple to a counterpart connector for example a luer connector of a syringe, a fluid pump or some other fluid store. A fluid communication path is therefore established between the connector and the interior cavity of catheter hub 4 such that fluid may flow bidirectionally between the patient and the fluid store via catheter hub 4 through sideport 4d and tubing 14. A clip 30 is slidably fitted along tubing 14 and may be used to clamp tubing 14 to occlude fluid flow if desired. To the underside of main body portion 4a are two wings 4e extending in opposite directions that may be used by the clinician to placed or fasten (by tape) the catheter hub to the skin of a patient. A catheter 10 extends from the distal portion 4b of catheter hub 4 by means of its proximal end 4b press fitted to the channel formed in distal portion 4b (FIGS. 3-4). A needle cannula or simply needle 12 extends through catheter hub 4 and is slidably inserted through catheter 10 so that its sharp distal tip 12a extends beyond the distal end 10a of catheter 10 as is conventionally known. Coupled to catheter hub 4 is a needle housing 6 that includes a body portion 6a with two side grippers 6b. Attached to the proximal end of needle housing 6 is a flash plug 8 that allows air to pass through so that flashed blood may be observed by the clinician to determine that the catheter and needle are properly placed into the vasculature, for example a vein of the patient. As shown in FIG. 1, catheter assembly 2 is in a ready to use position or a ready state.

FIG. 2 is a cross-sectional view of a major portion of catheter hub 4 and a proximal portion of needle housing 6 that is adjacent to the proximal end 4h of catheter hub 4. As shown, catheter hub 4 is formed by a wall 4f which inner wall 4f1 defines an internal or interior cavity 4g that extends along substantially the length of catheter hub 4. At main body portion 4a, a septum 18 is positioned proximal an opening 20 that forms the entrance to the bore of sideport 4d so that a through fluid path extends between tubing 14 and the interior cavity of catheter hub 4. For ease of discussion, the interior cavity 4g in catheter hub 4 distal septum 18 is designated 4g1 in the figures. Septum 18 may have an aperture 18a centered at the longitudinal axis 22 that extends along catheter hub 4. Given that septum 18 is made from an elastomeric material such as silicone or rubber, aperture 18a may not be needed. Instead, one or more slits may be provided to assist needle 12 to pass through septum 18. Positioned proximal septum 18 is a seal member 24 that, for the exemplar embodiment shown, may straddle main body portion 4a and proximal portion 4c of catheter hub 4.

Seal member 24 may also be made from an elastomeric material such as for example silicone or polyisoprene. A membrane 24a partitions seal member 24 into a distal section 24b and a proximal portion 24c. Although section 24c is shown to have a shorter length than distal section 24b, it may not necessarily be so. Seal member 24 is non-removably held to catheter hub 4 by coacting catch mechanisms at those components, for example a circumferential notch 24d at seal member 24 in catching contact with a circumferential protrusion 4j formed at inner wall 4f1 of catheter hub 4. Further formed in inner wall 4f1 of catheter hub 4 is another catch mechanism in the form of a circumferential groove 4i. Membrane 24a may have one or more slits (not shown) to assist needle 12 to more easily pierce or pass therethrough. Upon removal of needle 12, membrane 24a reseals so that chamber 24e formed by distal section 24b acts as a reservoir to store possible fluid back flowed from interior cavity 4g1 distal of septum 18.

A safety needle tip protector or needle clip 26 is also positioned inside catheter hub 4. Needle clip 26 has a base 26a that has an opening 26b that allows needle 12 to pass though. Extending from base 26a are two arms 26c1 and 26c2. For the exemplar embodiment shown, the base portion of needle clip 26 may include the funnel shaped portion where arms 26c1 and 26c2 incline from opposite edges of base 26a toward the longitudinal axis 22 and then level off from the lowest point of the incline in their natural position, as per shown in FIG. 4. The distal portions of arms 26c1 and 26c2 are bent away from the longitudinal axis 22 to form opposing bumps 26d1 and 26d2 and then bent toward the longitudinal axis 22 to form respective barriers 26e1 and 26e2 with curved end fingers. As shown in FIG. 4, at their natural position, barrier 26e2 overlaps barrier 26e1. Needle clip 26 may be made from a metallic material such as stainless steel.

As shown in FIGS. 2-3, barriers 26e1 and 26e2 are biased outwardly away from longitudinal axis 22 when needle 12 passes therebetween such that opposing bumps 26d1 and 26d2 are moved transversely away from longitudinal axis 22 to press against wall 24f at proximal section 24c of seal member 24. The pressed against portions of seal member 24 in turn push into circumferential groove 4i at the inner surface 4f1 of catheter hub 4 to fixedly hold needle clip 26 to catheter hub 4. Thus, when barriers 26e1 and 26e2 of needle clip 26 is being biased by needle 12, needle clip 26 and catheter hub 4 are fixedly coupled together. FIGS. 2-3 show catheter assembly 2 in the ready state.

Needle 12 has a needle feature 12b, shown as a notch in the exemplar embodiment, that has a dimension different from the rest of the needle such that the needle feature will not pass through the opening at base 26a of needle clip 26. For the embodiment in FIGS. 2-4, the opening at base 26a may be configured such that when the needle feature notch reaches the opening, the notch would be caught at an edge of the opening so that further proximal movement of needle 12 would pull needle clip 12 in the proximal direction relative to catheter hub 4. Notch 12b also acts to enable blood from the patient to flow into the needle and from there into the flash chamber of the needle housing so that the clinician can see that the needle and the catheter that superposes over the needle as conventionally known are correctly placed into the vein of the patient. It should be appreciated that the needle may have other needle features such as a bump or protuberance formed proximate to the sharp distal tip of the needle.

FIG. 4 shows needle 12 having been removed from catheter 10 and withdrawn in the proximal direction past septum 18 and membrane 24a of seal member 24. In addition, the distal tip 12a of needle 12 is shown captured inside needle clip 16 proximally from barriers 26e1 and 26e2. As barriers 26e1 and 26e2 are no longer biased by needle 12, arms 26c1 and 26c2 would return to their natural positions, i.e., in alignment along longitudinal axis 22, and barriers 26e1 and 26e2 overlappingly close down on each other to prevent distal tip 12a of needle 12 from further distal movement and exposure to the environment. In addition, with barriers 26e1 and 26e2 closing onto each other, opposing bumps 26d1 and 26d2 are released from wall 24f of seal member 24, such that needed clip 26 no longer frictionally attaches to catheter hub 4. At the same time, the needle feature 26b coacts against opening 12b as discussed above such that further proximal movement of needle 12 in the direction designated by directional arrow 28 would remove needle 12 and needle clip 26 from catheter hub 4.

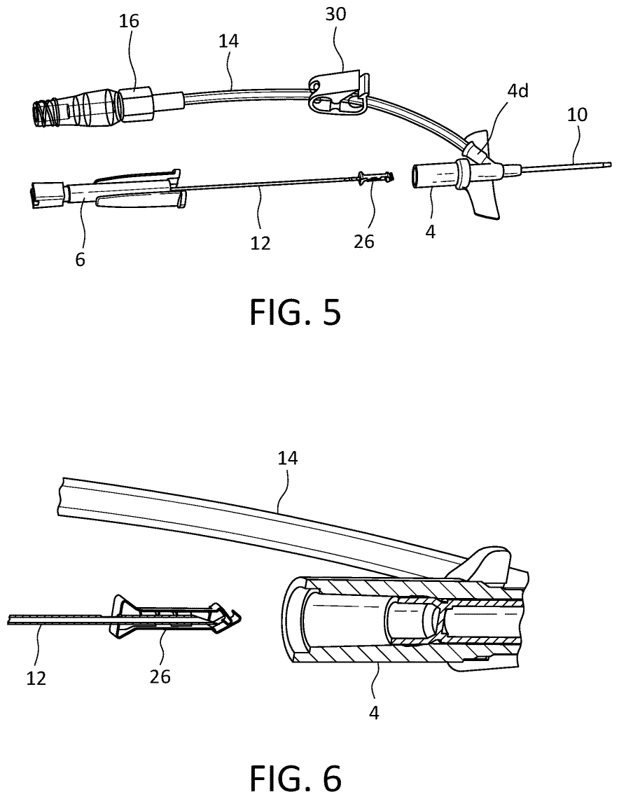

With arms 26c1 and 26c2 in their alignment positions along the longitudinal axis 22 as shown in FIG. 4, ribs 26f1 and 26f2 from arms 26c1 and 26c2, respectively, would come together to form a rib cage like structure to enclose the entrapped distal portion of needle 12 including distal tip 12a. FIGS. 5-6 show needle 12 and needle clip 26 being separated from catheter hub 4. With the distal tip 12a captured in needle clip 26, the catheter assembly, or more particularly the contaminated needle, may be considered to be in a safe position or a safe state.

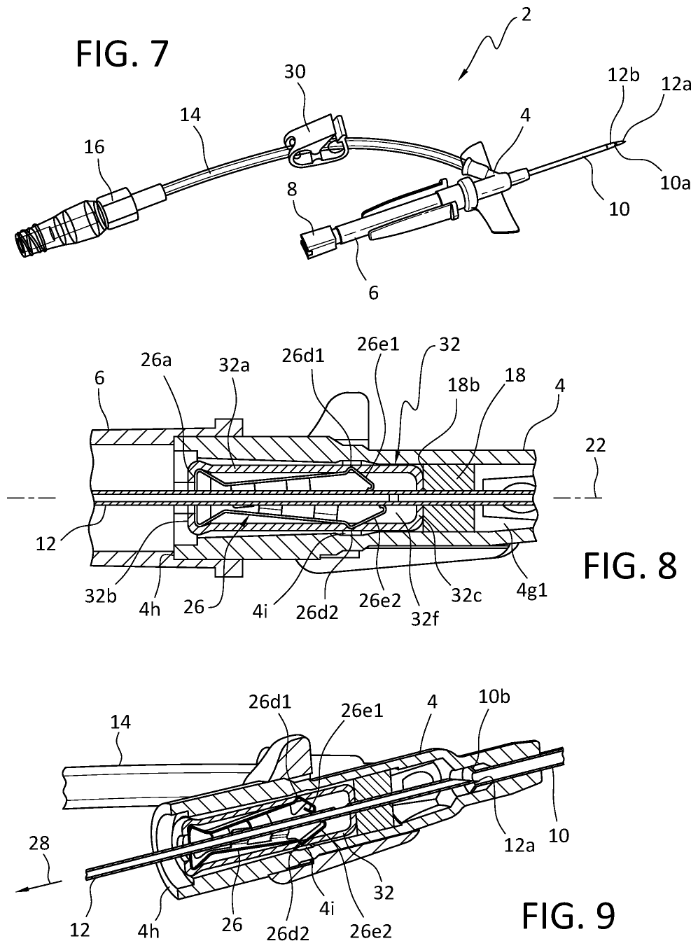

With reference to FIGS. 7-12, a second embodiment of the present invention is shown. For this embodiment and the later to be discussed embodiment, components that are the same or function the same as the embodiment shown in FIGS. 1-6 are labeled with the same reference numbers. An overall view of the catheter assembly 2 shown in FIG. 7 shows the overall length of catheter hub 4 may be less than the length of the catheter hub shown in the FIG. 1 embodiment. FIGS. 7-9 show the catheter assembly 2 in the ready state.

The exemplar embodiment shown in FIGS. 8-10 has needle clip 26 housed in a substantially cylindrical seal member 32 having a body 32a with a proximal end 32b and a front end 32c. For this embodiment, septum 18 is shown to have an aperture 18a that is aligned with an opening 32d at front end 32c of seal member 32. As seal member 32 is made of an elastomeric material such as for example silicone or polyisoprene, the distal tip of a needle can readily pierce therethrough, so that opening 32d may not be needed for seal member 32. Base 26a of needle clip 26 is attached, or in abutment with, proximal end 32b of seal member 32. An opening 32e may be provided at proximal end 32b of seal member 32 to enable needle 12 to pass into seal member 32. As shown in FIG. 8, in the ready state, needle 12 passes through seal member 32, septum 18 and extends through catheter 10 so that its distal tip 12a extends beyond distal end 10a of catheter 10. Front end 32c of seal member 32 is in abutment with the proximal surface 18b of septum 18, such that when the needle is withdrawn from the patient and passes into seal member 32, the fluid in interior cavity 4g1 of catheter hub 4 may back flow past septum 18 into the chamber 32f defined by wall 32a of seal member 32. Chamber 32 therefore acts as a reservoir for back flow fluid from interior cavity 4g1 when needle 12 is withdrawn from the patient. The coupling needle clip 26 with catheter hub 4 is similar to the earlier discussed embodiment in that when barriers 26e1 and 26e2 of needle clip 26 are biased by needle 12 passing therebetween, their respective bumps 26d1 and 28d2 would press corresponding portions of wall 32a of seal member 32 into circumferential groove 4i at the inner surface of catheter hub 4. As a result, needle clip 26 and seal member 32 are frictionally attached to the inner wall of catheter hub 4 in the ready state.

FIG. 9 shows needle 12 being withdrawn in the direction designated by directional arrow 28 relative to catheter hub 4 such that distal tip 12a is past proximal end 10b of catheter 10. As shown, barriers 26e1 and 26e2 continue to be biased by needle 12 so that seal member 32 and needle clip 26 housed therein continue to be frictionally held to the interior cavity of catheter hub 4.

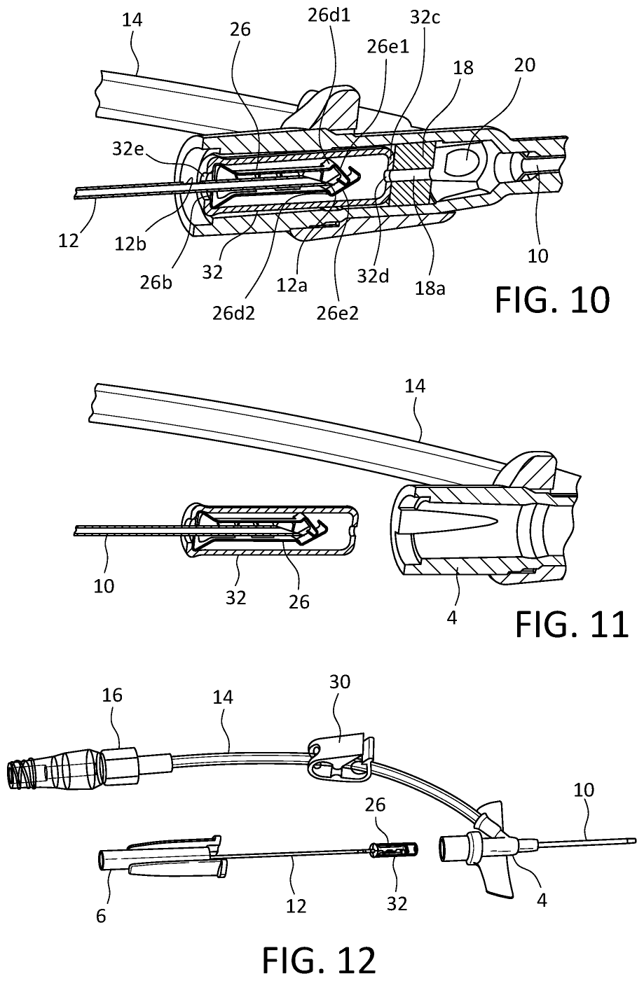

FIG. 10 shows distal tip 12a of needle 12 is captured in needle clip 26 proximal to barriers 26e1 and 26e2 such that the barriers, no longer biased by the needle, are in their natural overlapped position. Accordingly, opposing bumps 26d1 and 26d2 are released from seal member 32 so that seal member 32 and needle clip 26 are no longer frictionally held to catheter hub 4. For this embodiment, needle feature 12b is caught at base opening 26b so that further movement of needle 12 in the proximal direction removes seal member 32 and needle clip 26 housed therein from catheter hub 4, as per shown in FIG. 11. FIG. 12 shows an overall view where needle housing 6 is connected to needle clip 26 housed in seal member 32 by way of needle 12. In this embodiment, needle clip 26 is hidden from the environment from the ready state to when the used needle is disposed.

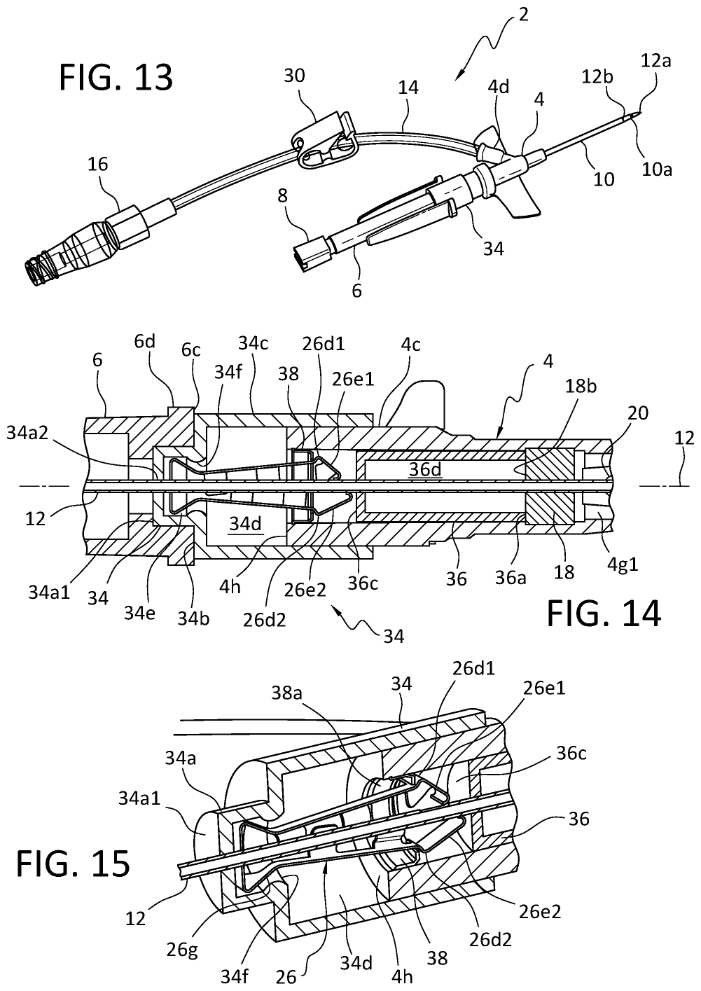

An overall view of catheter assembly 2 of the FIGS. 13-17 embodiment is illustrated in FIG. 13. Here a housing or collar 34 is shown at the distal end of needle housing 6. As best shown in FIGS. 14-15, needle housing 6 has a flange 6d at its distal end 6c for coupling to a counterpart connector for example a luer connector as is conventionally known. Collar 34 may be a cylindrical body having a hollow extension 34a from its proximal base 34b. Extension 34a has a proximal end 34a1 having an opening 34a2 to accommodate needle 12 passing therethrough. Base 34b of collar 34 is in abutment with distal end 6c of needle housing 6 in the ready state. Main body 34c of collar 34 forms a chamber 34d that expands into the space 34d defined by extension 34a. A circumferential protrusion 34f extends inwardly at the junction where main body 34c and extension 34a meet to form a narrowed opening between chamber 34d and space 34e. Needle clip 26 is non-removably held to collar 34 by means of its funnel-shaped base portion 26g being trapped in space 34e of extension 34a, as base portion 26g has a cross dimension larger than the narrowed opening formed by circumferential protrusion 34f.

As shown in FIGS. 14-16, inside catheter hub 4 a septum 18 is positioned proximal opening 20 to sideport 4d. A substantially sleeve like cylindrical elastomeric seal member 36 is positioned proximal septum 18, with its opened distal end 36a abutting the proximal surface 18b of septum 18. An opening or aperture 36b may be provided at proximal end 36c of seal member 36 to enable needle 12 to pass through. It should however be appreciated that an opening at proximal end 36c may not be needed insofar a needle can readily pierce through the elastomeric proximal end, particularly were at least one slit is provided thereat to assist the needle in piercing through the elastomeric material. A chamber 36d formed by seal member 36 may act as a reservoir to collect fluid that may back flow from interior cavity 4g1 through septum 18, when the needle is withdrawn proximally past septum 18.

Further shown in the exemplar embodiment of FIGS. 13-17 is a retainer member 38 that may be in the form of a snap ring that press fits to the opened proximal end of catheter hub 4 such that retainer member is non-removably attached to the opened proximal end of catheter hub 4. Retainer member 38 has an opening 38a that has a cross dimension that is smaller than the cross dimension of the transversely expanded opposing bumps 26d1 and 26d2 when barriers 26e1 and 26e2 are biased by needle 12 away from longitudinal axis 22. As a result, needle clip 26 is fixedly held to catheter hub 4 when barriers 26e1 and 26e2 are being biased by needle 12 in the ready state or when needle 12 is being withdrawn from the patient but its distal tip 12a is not yet entrapped in needle clip 26. When needle clip 26 is fixedly held to catheter hub 4, distal end 6c of needle housing 6 is in abutment contact with base 34b of collar 34. The length of needle 12 is such that its distal end 12a extends beyond distal end 10a of catheter 10 when the catheter assembly is in the ready state. Although not shown, an internal shoulder or step may be provided at the inner surface of collar 34 to coact against proximal end 4h of catheter hub 4 to prevent further distal movement of collar 34 relative to catheter hub 4 in the ready state, to assist in the insertion of the needle and its overlying catheter into the patient.

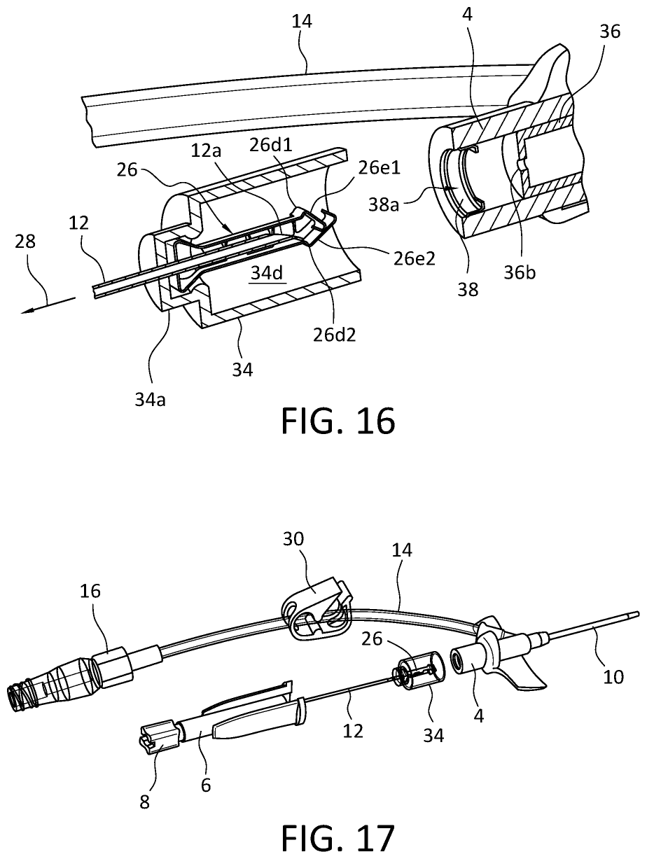

FIG. 16 shows collar 34 with needle clip 26 housed therein separated from catheter hub 4. As should be appreciated, once the distal tip 12a is entrapped inside needle clip 26, barriers 26e1 and 26e2 would return to their natural position since they are no longer biased by needle 12, thereby causing their associated opposing bumps 26d1 and 26d2 to move transversely toward longitudinal axis 22. As a result, the cross dimension between opposing bumps 26d1 and 26d2 decreases to be smaller than the cross dimension of opening 38a of retainer member 38 to allow needle clip 26 to be removed from retainer member 38 with further movement of needle 12 along the direction indicated by directional arrow 28. FIG. 17 shows an overall view of the catheter assembly 2 in the safe state where collar 34, having been separated from catheter hub 4, and its covered needle clip 26, which is connected to needle housing 6 via needle 12, may be safely disposed of.

It is the intension of the inventors that all matter described throughout this specification and shown in the accompanying drawings be interpreted as illustrative only and not in a limiting sense. Accordingly, it is intended that the invention be limited only by the spirit and scope of the hereto appended claims.

1. A catheter assembly comprising:
a catheter hub having a body defined by a wall having a distal end and an opened proximal end, and an interior cavity defined by an inner surface of the wall between the distal end and the opened proximal end;
a catheter having a distal end extending from the distal end of the catheter hub along a longitudinal axis to establish a passage to the interior cavity;
a septum positioned in the interior cavity of the catheter hub;
a needle housing having a needle hub and a distal portion having an opened distal end;
a needle having a proximal end attached to the needle hub and a sharp distal tip extending away from the distal end of the needle housing;
wherein the needle extends into the interior cavity of the catheter hub and slidably extends along the catheter with its distal tip extending beyond the distal end of the catheter and the distal portion of the needle housing adjacent to the proximal end of the catheter hub when the catheter assembly is in a ready state;
a needle tip protector slidably positioned along the needle, the needle tip protector having a base and opposing arms extending distally from the base to respective opposing apexes having corresponding opposing bumps and corresponding barriers downward of the apexes, the opposing bumps movable bi-directionally transversely to the longitudinal axis;
a sleeve member fixedly engaged to and in circumferential contact with the inner surface of the wall of the catheter hub;
wherein when the catheter assembly is in the ready state, the distal bumps of the opposing arms of the needle tip protector are positioned within the interior cavity of the catheter hub proximal to the septum, the needle passing between the barriers and biasing the barriers in a direction away from the longitudinal axis to cause the opposing bumps to be in frictional contact with the sleeve member; and
wherein when the needle is withdrawn from the catheter and the distal tip of the needle moves proximally past the barriers so that the barriers are no longer biased by the needle, the opposing bumps move away from the sleeve member toward the longitudinal axis to disengage the needle tip protector from the sleeve member such that the needle tip protector is separable from the catheter hub.

2. The catheter assembly of claim 1, wherein the sleeve member comprises a substantially cylindrical seal member having an inner surface defining a distal section and a proximal section separated by a membrane, the distal section having a distal end in abutment with a proximal surface of the septum, the proximal section having an opened proximal end for receiving at least a distal portion of the needle tip protector that includes the barriers and the opposing bumps such that when the catheter assembly is in the ready state, the opposing bumps are in frictional engagement with the inner surface of the proximal section of the seal member.

3. The catheter assembly of claim 1, wherein, in the ready state, the needle tip protector is housed in the interior cavity of the catheter hub proximal to the septum, a distal portion of the needle tip protector in frictional contact with a proximal section of the sleeve member such that the needle tip protector is non-removably held inside the interior cavity of the catheter hub.

4. The catheter assembly of claim 1, further comprising a side port hub integrally extending from the catheter hub, an opening at the wall of the catheter hub to the side port hub establishing a through passage between the side port hub and the interior cavity of the catheter hub, the side port hub having a non-patient opening adapted to be connected to a tubing;
wherein the septum is positioned in the interior cavity of the catheter hub proximal the side port opening.

5. The catheter assembly of claim 2, wherein the distal section of the seal member acts as a reservoir to contain back flow of blood when the needle is withdrawn from the septum and the membrane, and the membrane reseals as the distal tip of the needle is being captured in the needle tip protector to prevent blood from leaking into the interior cavity of the catheter hub.

6. The catheter assembly of claim 4, wherein the tubing has one end connected to the non-patient opening at the side port hub and an other end connected to a connector of a fluid store device.

7. The catheter assembly of claim 1, wherein the needle tip protector comprises a base having an opening for the needle to pass through, each of the arms having corresponding ribs folded orthogonally therefrom toward the other arm to form a rib cage like structure when the two arms are in substantial coplanar relationship with each other, one of the barriers overlapping the other of the barriers when the arms are in their natural unbiased positions.

8. The catheter assembly of claim 1, wherein the distal portion of the needle housing comprising an internal shoulder proximal from the opened distal end, the opened distal end receiving a proximal portion of the catheter hub with the proximal end of the catheter hub in abutment with the internal shoulder of the needle housing when the catheter assembly is in the ready state.

9. The catheter assembly of claim 1, further comprising wings extending from the catheter hub.

10. A catheter assembly comprising:
a catheter hub having a wall defining a body having an interior cavity defined by an inner surface of the wall between a distal end and an opened proximal end, a side port hub extending from the body, an opening at the wall connecting a passage in the side port hub with the interior cavity, a catheter having a distal end extending from the distal end of the catheter hub along a longitudinal axis to establish a fluid passage to the interior cavity;
a septum positioned in the interior cavity of the catheter hub proximal the opening at the wall;
a needle housing having a needle hub and a distal portion having an opened distal end;
a needle having a proximal end attached to the needle hub and a sharp distal tip extending away from the distal end of the needle housing, the needle extending into the interior cavity of the catheter hub, piercing past the septum and slidably extends along the catheter with its distal tip extending beyond the distal end of the catheter in a ready state;
a sleeve member fixedly engaged to and in circumferential contact with the inner surface of the wall of the catheter hub, a distal end of the sleeve member in contact with a proximal surface of the septum;
a needle tip protector slidably positioned along the needle, the needle tip protector having a base and opposing arms extending distally from the base to respective opposing apexes having corresponding opposing bumps and corresponding barriers transverse of the apexes toward the longitudinal axis, the opposing bumps movable bi-directionally transversely to the longitudinal axis;
wherein, when the catheter assembly is in the ready state, the distal bumps of the opposing arms of the needle tip protector are positioned within the sleeve member, the needle passing between the barriers and biasing the barriers in a direction away from the longitudinal axis such that the opposing bumps are biased into frictional contact with the sleeve member.

11. The catheter assembly of claim 10, wherein when the needle is withdrawn from the catheter and the distal tip of the needle moves proximally past the barriers so that the barriers are no longer biased by the needle, the opposing bumps move away from the sleeve member toward the longitudinal axis to disengage the needle tip protector from the sleeve member such that the needle tip protector is separable from the catheter hub.

12. The catheter assembly of claim 10, wherein the distal portion of the needle housing is adjacent to the proximal end of the catheter hub when the catheter assembly is in a ready state.

13. The catheter assembly of claim 10, wherein the sleeve member comprises a substantially cylindrical seal member having an inner surface defining a distal section having the distal end and a proximal section, a membrane separating the distal section and the proximal section, the proximal section having an opened proximal end for receiving at least a distal portion of the needle tip protector that includes the barriers and the opposing bumps such that when the catheter assembly is in the ready state, the opposing bumps are in frictional engagement with the inner surface of the proximal section of the seal member.

14. The catheter assembly of claim 10, wherein, in the ready state, the needle tip protector is housed in the interior cavity of the catheter hub proximal to the septum, a distal portion of the needle tip protector in frictional contact with a proximal section of the sleeve member such that the needle tip protector is non-removably held inside the interior cavity of the catheter hub.

15. The catheter assembly of claim 13, wherein the distal section of the seal member acts as a reservoir to contain back flow of blood when the needle is withdrawn from the septum and the membrane, and the membrane reseals as the distal tip of the needle is being captured in the needle tip protector to prevent blood from leaking into the interior cavity of the catheter hub.

16. The catheter assembly of claim 10, further comprising a tubing having one end fluidly connected to passage of the side port hub and an other end connected to a connector of a fluid store device.

17. The catheter assembly of claim 10, wherein the needle tip protector comprises a base having an opening for the needle to pass through, each of the arms having corresponding ribs folded orthogonally therefrom toward the other arm to form a rib cage like structure when the two arms are in substantial coplanar relationship with each other, one of the barriers overlapping the other of the barriers when the arms are in their natural unbiased positions.

18. The catheter assembly of claim 1, wherein the distal portion of the needle housing comprising an internal shoulder proximal from the opened distal end, the opened distal end receiving a proximal portion of the catheter hub with the proximal end of the catheter hub in abutment with the internal shoulder of the needle housing when the catheter assembly is in the ready state.
